(19) United States
(12) Patent Application Publication
(10) Pub. No.: US 2022/0280125 A1
(43) Pub. Date: Sep. 8, 2022

(54) X-RAY BED

(71) Applicant: CARESTREAM HEALTH, INC., Rochester, NY (US)

(72) Inventors: Xiaohui WANG, Pittsford, NY (US); Robert S. JONES, Rochester, NY (US)

(21) Appl. No.: 17/666,848

(22) Filed: Feb. 8, 2022

Related U.S. Application Data
(60) Provisional application No. 63/155,405, filed on Mar. 2, 2021.

(51) Int. Cl. A61B 6/04; A61B 6/00

(52) U.S. Cl. CPC A61B 6/0407; A61B 6/4429; A61B 6/548

(57) ABSTRACT

A radiography system includes a bed, having a surface for receiving and supporting a patient lying thereon, and an x-ray source positioned below the patient. A digital x-ray detector is positioned above the patient, facing the x-ray source, whereby the patient is positioned between the x-ray source and the detector. This configuration enables capturing an image of the patient exposed by the x-ray source.

### CROSS REFERENCE TO RELATED APPLICATIONS

This application claims priority to U.S. Patent Application Ser. No. 63/155,405, filed Mar. 2, 2021, in the name of Wang et al., and entitled X-RAY TUBE UNDER PATIENT BED IN INTENSIVE CARE UNIT, which is hereby incorporated by reference herein in its entirety.

### BACKGROUND OF THE INVENTION

The subject matter disclosed herein relates to a medical digital x-ray imaging system incorporated into a patient bed.

Infection control has emerged as being among the most critically important factors in healthcare delivery as a result of the global propagation of Covid 19. One consequence of the pandemic is that it has accelerated the innovation process in a multitude of healthcare product arenas, including in areas such as personal protective equipment, ventilators, assays for Covid 19 testing, and antimicrobials, among many others. In this regard, of great interest is how to build better infection control into the medical imaging process in the context of portable X-ray imaging of patients in intensive care units, in emergency departments, and other medical facility areas. An approach for radiographic imaging disclosed herein represents a transformational shift in the radiographic imaging process because it does not involve transporting and positioning mobile x-ray units for imaging patient. This allows patients afflicted with infectious disease to remain in isolation from staff that would normally perform the radiographic imaging at the patient bedside. It also brings the benefit of posterior-anterior x-ray projection images of the chest to bedside imaging.

The discussion above is merely provided for general background information and is not intended to be used as an aid in determining the scope of the claimed subject matter.

### BRIEF DESCRIPTION OF THE INVENTION

A radiography system includes a bed having a surface for receiving and supporting a patient and an x-ray tube positioned below the patient. A digital x-ray detector is positioned above the patient, facing the x-ray tube, to capture an image of the patient exposed by the x-ray tube. An advantage that may be realized in the practice of some disclosed embodiments of the x-ray bed includes infectious disease isolation and bedside posterior-anterior x-ray imaging.

In one embodiment, a radiography system includes a bed having a surface for receiving and supporting a patient, an x-ray source positioned below the patient, and a digital x-ray detector positioned above the patient to capture an image exposed by the x-ray source.

In one embodiment, a method includes the steps of attaching an x-ray tube to a portion of a bed below the patient, and attaching a digital radiographic detector to a portion of the bed so that the detector is positioned above the patient and facing the x-ray tube.

The system makes use of several components. First, the x-ray tube and generator are installed in the patient bed under the patient. During x-ray imaging, the x-ray tube is positioned under the patient at the desired SID and aligned with the patient anatomy. Second, the x-ray detector is deployed above the patient and aligned with the x-ray beam coverage. The positioning of the x-ray tube and the detector may be performed automatically based on information from a camera installed in the medical facility near the patient bed. Activating an x-ray exposure using the x-ray tube can be initiated remotely outside the patient room in order to separate hospital staff from the patient, who is in isolation, in order to maximize the protection of the hospital staff from any infectious disease. Post acquisition images are also available for review remotely outside the patient room on a network connected display.

The summary descriptions above are not meant to describe individual separate embodiments whose elements are not interchangeable. In fact, many of the elements described as related to a particular embodiment can be used together with, and possibly interchanged with, elements of other described embodiments. Many changes and modifications may be made within the scope of the present invention without departing from the spirit thereof, and the invention includes all such modifications.

This brief description of the invention is intended only to provide a brief overview of subject matter disclosed herein according to one or more illustrative embodiments, and does not serve as a guide to interpreting the claims or to define or limit the scope of the invention, which is defined only by the appended claims. This brief description is provided to introduce an illustrative selection of concepts in a simplified form that are further described below in the detailed description. This brief description is not intended to identify key features or essential features of the claimed subject matter, nor is it intended to be used as an aid in determining the scope of the claimed subject matter. The claimed subject matter is not limited to implementations that solve any or all disadvantages noted in the background.

### BRIEF DESCRIPTION OF THE DRAWINGS

So that the manner in which the features of the invention can be understood, a detailed description of the invention may be had by reference to certain embodiments, some of which are illustrated in the accompanying drawings. It is to be noted, however, that the drawings illustrate only certain embodiments of this invention and are therefore not to be considered limiting of its scope, for the scope of the invention encompasses other equally effective embodiments. The drawings below are intended to be drawn neither to any precise scale with respect to relative size, angular relationship, relative position, or timing relationship, nor to any combinational relationship with respect to interchangeability, substitution, or representation of a required implementation, emphasis generally being placed upon illustrating the features of certain embodiments of the invention. In the drawings, like numerals are used to indicate like parts throughout the various views. Thus, for further understanding of the invention, reference can be made to the following detailed description, read in connection with the drawings in which:

FIG. 1 is a perspective view of a patient lying on a bed equipped with a radiographic imaging system; and

FIG. 2 is a side view of FIG. 1;

FIG. 3 is a system view of FIG. 1;

FIG. 4 is an exemplary display for a remote monitor;

FIG. 5 is a schematic diagram of a detector movement assembly; and

FIG. 6 is a schematic diagram of the x-ray tube head adjustment assembly.

### DETAILED DESCRIPTION OF THE INVENTION

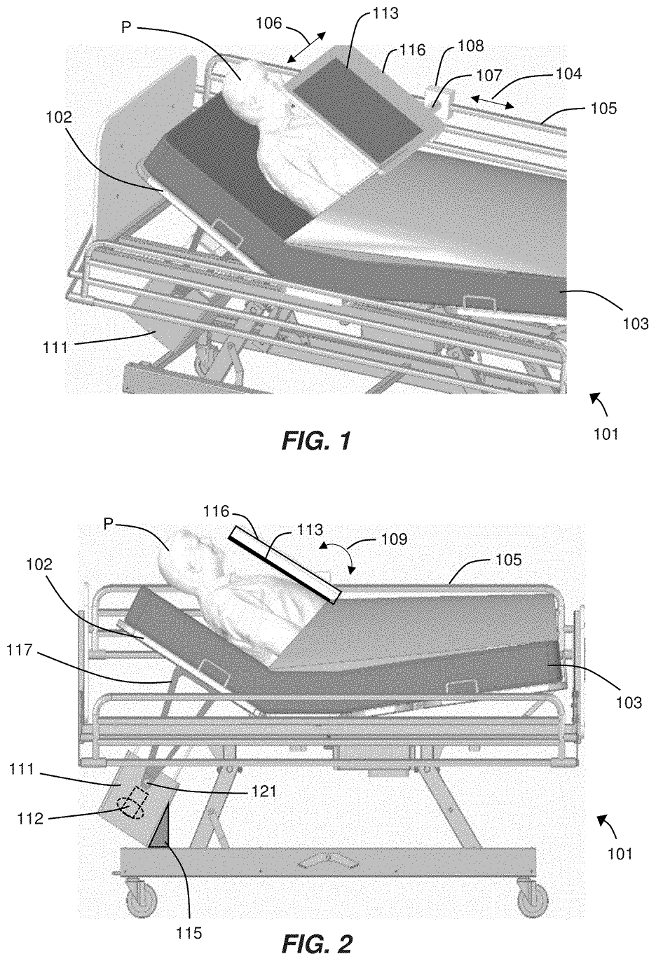

With reference to FIGS. 1 and 2, there is illustrated a radiography system configured to be incorporated into a patient bed 101 to capture radiographic images of a patient P lying on the patient bed 101. The patient bed 101 may include a cushion 103 having a surface for supporting the patient P lying thereon. The patient bed 101 may include an angularly adjustable section 102 for raising and lowering a portion of the body of the patient P. The adjustable section 102 may be tilted at various angles for purposes of patient x-ray imaging or for patient comfort. The radiographic imaging system includes an x-ray tube 111 and a digital radiographic detector 113 both attached to different portions of the patient bed 101. The x-ray tube 111 is attached to a portion 102 of the patient bed below the patient P. The x-ray tube 111 is aimed upward toward a wireless digital radiographic detector 113 positioned above the patient P and facing the x-ray tube 111. Thus, the patient P is suitably positioned between the x-ray tube 111 and the detector 113 in order for the radiographic imaging system to capture a radiographic image of a portion of the patient. The materials of the patient bed that may be positioned between the x-ray tube 111 and the detector 113 may be selected for sufficient radiolucency to allow capturing radiographic images of the patient P with image quality suitable for diagnostic purposes.

The detector 113 may be secured in a detector holder 116. The detector holder 116 may be attached to a railing 105 of the patent bed using a motorized base 108, and an extendable rotatable rigid support arm 107, which altogether may be referred to as the detector movement assembly. The extendable rotatable rigid support arm 107 and the detector 113 are moved along the rail 105 in either of directions 104 using the motorized base 108 under remote operator control in order to position the detector 113 as desired. The detector holder 116 may be attached to the motorized base 108 by a motorized extendable and rotatable rigid support arm 107 which may be used to move the detector 113 sideways along directions 106, i.e., to the left and to the right of the patient P, or to rotate the detector 113 in directions 109 about an axis of the support arm 107 as described herein below.

In one embodiment, the x-ray tube 111 may be attached to the adjustable section 102 of the patient bed 101 using a rigid attachment frame 117. In this embodiment, the attachment frame 117 and the tube head 111 move together with the angularly adjustable section 102 of the patient bed 101. Thus, the x-ray tube 111 may maintain a constant distance relative to the adjustable section 102 of the patient bed 101 when the adjustable section 102 is tilted. The x-ray tube 111 may be configured to rotate about axis 121 where the tube head 111 is attached to the attachment frame 117. In one embodiment, the x-ray tube 111 may be attached only to a stationary base frame 115 of the patient bed. In this embodiment, the stationary base frame 115 and the tube head 111 remain stationary while the adjustable section 102 of the patient bed 101 is moved. The x-ray tube 111 may be movable closer to and further from the detector 113 along base frame 115 under remote operator control as described herein below. Tube head 111 includes an x-ray source 112, which may include a cold cathode x-ray source or a carbon nanotube x-ray source to emit x-rays through patient P toward detector 113.

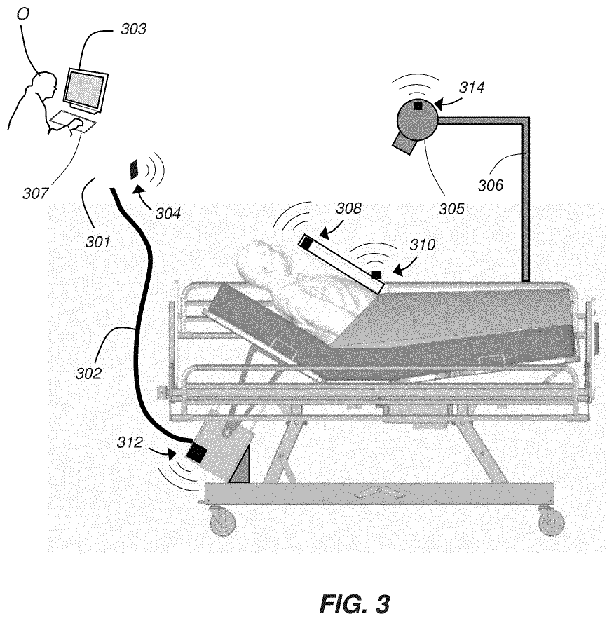

With reference to FIG. 3, additional components of the radiographic imaging system of the present invention are illustrated. An operator control console 301 may include a processing system and electronic memory for controlling imaging operations of the x-ray source 112 in the tube head 111 and the detector 113. The control console 301 may communicate over a cable 302 connected to the x-ray tube 111 and/or the detector 113, or may communicate wirelessly with components of the radiographic imaging system described herein. Wireless transceivers 304, 308, 310, 312, and 314 may be provided in a control console 310, the detector 113, the motorized base 108, the tube head 111, and a video camera 305, respectively. Thus, the control console may be configured to transmit wireless control signals using transceiver 304, in response to operator O instructions input to the control console 301, to synchronize image capture timing in the detector 113 and to receive radiographic images captured and transmitted by the detector 113. Similarly, the control console may be configured to transmit control signals to transceiver 310 in the motorized base 108 to position the detector 113, as desired, and to transceiver 312 to control power levels and to activate firing of the x-ray source 112. The control console 301 may execute programs to control firing of the x-ray source 112 in tube head 111 and to control timing of an image capture procedure by the detector 113. The control console 301 may also be configured to transmit wireless control signals to control movement of the motorized base 108 and extension and retraction and rotation of the support arm 107, in response to input operator requests, for positioning the detector 113 as desired. The control console 301 may receive instructions and commands from an operator O inputting requests via a keyboard 307 or mouse, for example. The control console 301 includes a connected monitor 303 for displaying communications from the radiographic imaging system and performance status of operator requests. In one embodiment, the monitor 303 is a touch screen monitor displaying a graphical user interface for receiving operator inputs as described herein. A video camera 305 may be positioned proximate the patient P for use in remote x-ray imaging of the patient P. In one embodiment, the video camera 305 may be attached to the patient bed 101 using a support arm 306, or it may be attached to another structure in a room of a medical facility treating the patient P. The camera 305 may be configured to wirelessly transmit video images of the patient P to the control console 301 via transceiver 314. The detector 113 and tube head 111 may include sensors for transmitting relative spatial orientation coordinates to allow the radiographic imaging system to determine proper alignment of the x-ray source 112 and detector 113.

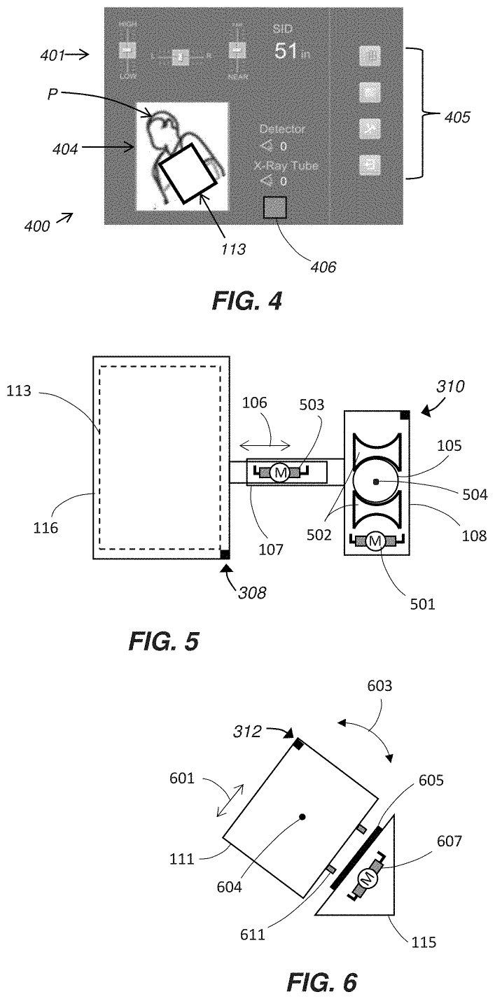

FIG. 4 illustrates one embodiment of a display configuration 400 shown on the screen of the monitor 303 for viewing by an operator O using the control console 301. The display may provide operator controls to assist the operator O to remotely control imaging procedures performed by the radiographic imaging system described herein. A portion 404 of the display 400 includes a video feed from the video camera 305 to enable the operator O to verify and, if necessary, adjust a position of the detector 113 relative to the patient P, for example. In one embodiment, the monitor 303 is a touch screen monitor, providing slider controls 401 configured to adjust a position of the detector 113 and the tube head 111, as described herein, in response to the operator manipulating the sliders. Another set of control features 405 allow the operator O to activate the x-ray source 112 and/or the detector 113, for example. The display 400 may be used to display a calculated SID measured by using a position of the tube head 111 on support base 115. Displayed controls may be selected by the operator to selectively display a different set of functions 405 to control other parameters of the radiographic imaging system as described herein, and to display other numerical data fields as part of a touch screen implementation for operator control of the radiographic imaging system. An operator may use remote positioning controls 401 while viewing the live video of patient P on camera display 404 in order to properly position the detector 113, for example. The operator can also use remote positioning controls 401 to move and align the tube head 111 into a proper orientation with detector 113 using, for example, the numerical display of the source to image distance (SID). The x-ray source 112 in the tube head 111 may be controllably fired, using source firing control 406, to capture radiographic images of the patient P in the DR detector 113.

FIG. 5 illustrates a schematic diagram of the detector movement assembly. The detector 113 may be interchangeably secured in the detector holder 116. The detector holder 116 is attached to extendable and rotatable support arm 107 which, in one embodiment, may be a telescopic arm that is configured to be extended and retracted along directions 106. The support arm may also be rotated in directions 109 by the motor 503. Both the extension/retraction and rotation movements may be performed under control of the motor 503 which receives control signals transmitted by control console 301 to transceiver 310 in motorized base 108 which are then relayed to the motor 503. The support arm 107 is attached to movable motorized base 108 which may be configured to be moved along rail 105 parallel to rail axis 504 by a motor 501 driving a pair of rollers 502 contacting the rail 105 on opposite sides thereof under control of signals transmitted by control console 301 to transceiver 310 in motorized base 108 which are then relayed to the motor 501. The motor 501 may drive the pair of rollers 502 in either of two opposite rotational directions to move the motorized base, as well as the detector 113 in the detector holder 116, in either of directions 104.

FIG. 6 illustrates a schematic diagram of a tube head adjustment assembly. The x-ray tube head 111 may be attached to a positioning plate 605 using posts 611 secured to the tube head 111 and inserted into the positioning plate 605, which is attached to an angled surface of the stationary base frame 115. The positioning plate 605 is configured to rotate the tube head 111 about a rotation axis 604 and to translate the tube head 111 parallel to the angled surface of the stationary base frame 115 under control of the motor 607 which receives control signals transmitted from the control console 301 to the transceiver 312 in the tube head 111 which are then relayed to the motor 607. By the operator O selectively controlling movement of the positioning plate 605 the tube head 111 may be rotated in either of directions 603 and moved linearly in directions 601 closer to or further from the detector 113.

As will be appreciated by one skilled in the art, aspects of the present invention may be embodied as a system, method, or computer program product. Accordingly, aspects of the present invention may take the form of an entirely hardware embodiment, an entirely software embodiment (including firmware, resident software, micro-code, etc.), or an embodiment combining software and hardware aspects that may all generally be referred to herein as a “service,” “circuit,” “circuitry,” “module,” and/or “system.” Furthermore, aspects of the present invention may take the form of a computer program product embodied in one or more computer readable medium(s) having computer readable program code embodied thereon.

Any combination of one or more computer readable medium(s) may be utilized. The computer readable medium may be a computer readable signal medium or a computer readable storage medium. A computer readable storage medium may be, for example, but not limited to, an electronic, magnetic, optical, electromagnetic, infrared, or semiconductor system, apparatus, or device, or any suitable combination of the foregoing. More specific examples (a non-exhaustive list) of the computer readable storage medium would include the following: an electrical connection having one or more wires, a portable computer diskette, a hard disk, a random access memory (RAM), a read-only memory (ROM), an erasable programmable read-only memory (EPROM or Flash memory), an optical fiber, a portable compact disc read-only memory (CD-ROM), an optical storage device, a magnetic storage device, or any suitable combination of the foregoing. In the context of this document, a computer readable storage medium may be any tangible medium that can contain, or store a program for use by or in connection with an instruction execution system, apparatus, or device.

Program code and/or executable instructions embodied on a computer readable medium may be transmitted using any appropriate medium, including but not limited to wireless, wireline, optical fiber cable, RF, etc., or any suitable combination of the foregoing.

Computer program code for carrying out operations for aspects of the present invention may be written in any combination of one or more programming languages, including an object oriented programming language such as Java, Smalltalk, C++ or the like and conventional procedural programming languages, such as the “C” programming language or similar programming languages. The program code may execute entirely on the user's computer (device), partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider).

Aspects of the present invention are described herein with reference to flowchart illustrations and/or block diagrams of methods, apparatus (systems) and computer program products according to embodiments of the invention. It will be understood that each block of the flowchart illustrations and/or block diagrams, and combinations of blocks in the flowchart illustrations and/or block diagrams, can be implemented by computer program instructions. These computer program instructions may be provided to a processor of a general purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks.

These computer program instructions may also be stored in a computer readable medium that can direct a computer, other programmable data processing apparatus, or other devices to function in a particular manner, such that the instructions stored in the computer readable medium produce an article of manufacture including instructions which implement the function/act specified in the flowchart and/or block diagram block or blocks.

The computer program instructions may also be loaded onto a computer, other programmable data processing apparatus, or other devices to cause a series of operational steps to be performed on the computer, other programmable apparatus or other devices to produce a computer implemented process such that the instructions which execute on the computer or other programmable apparatus provide processes for implementing the functions/acts specified in the flowchart and/or block diagram block or blocks.

This written description uses examples to disclose the invention, including the best mode, and also to enable any person skilled in the art to practice the invention, including making and using any devices or systems and performing any incorporated methods. The patentable scope of the invention is defined by the claims, and may include other examples that occur to those skilled in the art. Such other examples are intended to be within the scope of the claims if they have structural elements that do not differ from the literal language of the claims, or if they include equivalent structural elements with insubstantial differences from the literal language of the claims.

What is claimed is:

1. A radiography system comprising:
a patient bed having a surface for receiving and supporting a patient;
an x-ray source attached to the patient bed and positioned below the patient; and
a digital x-ray detector attached to the patient bed and positioned above the patient, the digital x-ray detector configured to capture an image of the patient exposed by the x-ray source.

2. The system of claim 1, wherein the patient bed is configured to receive and support a patient positioned between the x-ray source and the digital x-ray detector.

3. The system of claim 2, further comprising a rigid x-ray support arm attached to a movable portion of the patient bed and to the x-ray source, the x-ray support arm and the x-ray source configured to move together with the movable portion of the patient bed.

4. The system of claim 2, further comprising an x-ray support base attached to a stationary portion of the patient bed and to the x-ray source, the x-ray support base configured to move the x-ray source closer to or further from the patient.

5. The system of claim 3, further comprising a rigid detector support arm attached to the patient bed and to the digital x-ray detector, the rigid detector support arm configured to move the digital x-ray detector from side-to-side and to tilt the digital x-ray detector.

6. The system of claim 5, wherein the detector support arm is configured to be remotely controlled to move the digital x-ray detector into a position for capturing a radiographic image of the patient.

7. The system of claim 6, further comprising a digital camera configured to transmit an image of the detector and the patient.

8. The system of claim 7, wherein the x-ray source comprises a cold cathode tube or a carbon nanotube.

9. A method comprising:
attaching an x-ray source to a portion of a bed, the bed comprising a cushion, the x-ray source positioned below the cushion, wherein the cushion is configured to support a patient lying thereon; and
attaching a digital radiographic detector to a portion of the bed, wherein the detector is positioned above the cushion and facing the x-ray source.

10. The method of claim 9, further comprising placing a patient on the cushion and activating the x-ray source to capture a radiographic image of the patient in the digital detector.

11. The method of claim 10, further comprising positioning a camera proximate the patient and transmitting a video image of the detector and the patient to a remote monitor.

12. A radiography system comprising:
a patient bed having a surface for receiving and supporting a patient;
an x-ray source attached to the patient bed, the x-ray source positioned below the surface; and
a digital x-ray detector attached to the patient bed for capturing an image of the patient exposed by the x-ray source, the detector positioned above the surface and facing the x-ray source.

13. The system of claim 12, wherein the patient bed is configured to receive and support a patient between the x-ray source and the digital x-ray detector.

14. The system of claim 13, further comprising a rigid x-ray support arm attached to a movable portion of the patient bed and to the x-ray source, the x-ray support arm configured to secure at a fixed distance the x-ray source relative to the movable portion of the patient bed.

15. The system of claim 13, further comprising a stationary x-ray support base attached to the patient bed and configured to translate the x-ray source closer to or further from the detector.

16. The system of claim 14, wherein the rigid x-ray support arm is attached to a tube head containing the x-ray source and is configured to rotate the tube head.

17. The system of claim 16, further comprising a detector support arm, wherein the x-ray support arm and the detector support arm are configured to movably align the x-ray source and the digital x-ray detector for capturing a radiographic image of the patient.

18. The system of claim 13, further comprising a digital camera configured to transmit a video image of the detector and the patient.
